(19) United States
(12) Patent Application Publication
(10) Pub. No.: US 2018/0265900 A1
(43) Pub. Date: Sep. 20, 2018

(54) PROCESS AND METHOD FOR SIMULTANEOUS SACCHARIFICATION AND FERMENTATION USING MICROALGAE

(71) Applicant: BIOPROCESS ALGAE, Omaha, NE (US)

(72) Inventors: Toby Ahrens, Bethesda, MD (US); Adam CROTTEAU, Omaha, NE (US); Christopher MALONEY, Walpole, ME (US); Thiruvenkadam VISWANATHAN, Omaha, NE (US)

(21) Appl. No.: 15/544,503

(22) PCT Filed: Jan. 19, 2016
(86) PCT No.: PCT/US16/13923

§ 371 Date: Jul. 18, 2017

Related U.S. Application Data
(60) Provisional application No. 62/105,405, filed on Jan. 20, 2015.

(51) Int. Cl. C12P 7/10; C12N 9/26

(52) U.S. Cl. CPC C12P 7/10; C12P 2201/00; C12P 2203/00; C12N 9/2411

(57) ABSTRACT

The present invention generally relates to the production of biofuels and, in particular, to a process for simultaneous saccharification and fermentation using a microalgae substrate. According to one aspect of the present invention, a process is provided in which the temperature and pH of a broth mixture are adjusted to slow the rate of glucose conversion and to match the glucose metabolizing rate of the microalgae.

### FIELD OF THE INVENTION

The present invention generally relates to the production of algae for food, feed, chemicals and biofuels and, in particular, to a process for simultaneous saccharification and fermentation using microalgae as a substrate.

### BACKGROUND OF THE INVENTION

The use of microalgae to produce biofuels has shown significant promise. However, the cost of producing and fermenting using microalgae has remained high.

In the conventional production of ethanol-based biofuels, starch is converted to sugar via the enzymatic conversion process. In this process, specialized enzymes catalyze the depolymerization of starch into glucose. This step is alternatively referred to as enzymatic hydrolysis or saccharification. Many advanced biofuels, such as cellulosic ethanol production, use similar processes that employ specialized enzymes to break down cellulose, hemi-cellulose and lignin into fermentable sugars. After saccharification, the released glucose is fermented to ethanol using a substrate such as yeast, bacteria or microalgae. Thereafter, the ethanol is separated from the aqueous fermentation broth and is available to mix with gasoline.

At an industrial scale, saccharification is usually carried out using batch or fed-batch reactors. In a batch process, all components are placed in the reactor at the beginning of the hydrolysis with no further input or outputs to the reactor. In a fed-batch process, substrate components are periodically added in order to control the reaction rate. For example, enzymes or glucose additions are made to balance the feed rate of the substrate.

In the process of creating biofuels using microalgae, “heterotrophic” fermentation is used to convert sugars to biomass and oil which can be converted into biofuels. In this process, sugars typically represent the largest and most expensive component required by fermentations.

For microalgae, a constant, steady source of carbon (usually glucose) is a necessary component in the fermentation process. Typically, the glucose is provided by continuous dosing of pre-saccharified monomers (e.g. glucose derived from starch). Pre-saccharified monomers (e.g. glucose from starch) are typically produced from conversion from starch via enzymatic saccharification or acid hydrolysis, followed by a combination of filtration, clarification, refining and evaporation/concentration steps, each of which adds cost to the sugar feedstock. Simultaneous saccharification and fermentation (SSF) can reduce these processing costs by producing and utilizing the glucose in the same vessel, eliminating the need for concentration, processing and handling of glucose generated in a separate vessel and location. Further, while this SSF approach has been used for faster growing organisms such as Saccharomyces spp. in ethanol fermentation, the typical saccharification step proceeds too rapidly for algal fermentations, resulting in inhibitory levels of the sugar monomer released into the fermentation broth.

Because of the inhibitory effects of high glucose levels, dosing during fermentation usually requires careful glucose monitoring, changes to dosing rate, and sterilizations of the enzymes and glucose to be added.

The primary advantage of the fed-batch operation is the ability to control reaction rates by making additions of materials (i.e. usually sugar feedstocks). To balance reactions, predetermined feed profiles are used along with operational experience (i.e. trial and error). This approach is uneven and subject to variations depending on the skill of the operator and the repeatability of the conditions and ingredients. Automation of the fed batch dosing process can add cost.

Regardless of the approach, conventional optimization methods are often designed to maximize the rate of saccharification and the rate of sugar conversion by a given substrate. For microalgae, maximizing these rates reduces algal productivity since high glucose levels cause the metabolism rate of the microalgae to decline. As a result, fermentation using microalgae is very labor intensive and requires continuous and repeated rebalancing of glucose conditions during processing. Ultimately, the cost-benefit of using microalgae as a substrate is significantly reduced.

### SUMMARY OF THE DISCLOSURE

To minimize the limitations found in the prior art, and to minimize other limitations that will be apparent upon the reading of the specifications, the preferred embodiment of the present invention provides a novel process in which unsaccharified sugars are added at the beginning of a fermentation and the saccharifying enzyme activity is controlled by temperature, pH, or enzyme concentration in the broth mixture to control the rate of glucose conversion and to match the glucose metabolizing rate of microalgae. In use, the present invention addresses several primary technical problems in fermentation technologies: lowering the cost of the sugar feedstock, simplifying unit operations with fewer process steps, and improved contamination control.

According to one aspect of the present invention, a process is provided which reduces the cost of the sugar feedstock by enabling the use of carbohydrate polymers prior to complete saccharification. Pre-saccharified monomers (e.g. glucose from starch) typically require conversion from starch via enzymatic saccharification or acid hydrolysis, followed by a combination of filtration, clarification, refining and evaporation/concentration steps, each of which adds cost to the sugar feedstock. The process disclosed herein enables use of the carbohydrate polymers in the SSF process, eliminating the costs associated with additional process steps.

According to a preferred embodiment of the present invention, a preferred method of the present invention includes selection of a species of microalgae for use in fermentation. Preferably, the selected alga is tolerant to high concentrations of unconverted sugars. Thereafter, the microalgae are combined with unconverted sugars (preferably in the form corn mash or the like) within a fermentation vessel. Thereafter, enzymes are added which convert the unconverted sugars via enzymatic saccharification into glucose. The glucose is then simultaneously made available to the algae for fermentation. During this process, the glucose release rate is preferably controlled and matched to the target glucose levels in the fermentation broth for the selected species of microalgae as discussed further below.

According to a preferred embodiment, the sugar kinetics (i.e. rates of sugar release) are controlled via adjustments to the pH and temperature of the broth mixture.

According to an alternative preferred embodiment, the sugar release kinetics are matched with external nitrogen (‘N’) supplementation to balance the fermentation at different carbon (‘C’):N ratios in-situ (with enzymes, for example) and augmenting it with N supplements at specific times.

The process disclosed herein allows for optimum alga performance while eliminating or reducing the need for sugar or enzyme additions as the fermentation progresses. Benefits of using this approach include operational simplicity with improved organism performance (fewer additions over the course of a fermentation, and in some cases, true batch operation that acts as a fed-batch system through controlled release of sugars in-situ) as well as lower contamination risk (fewer additions).

The process disclosed herein also simplifies unit operations by reducing or eliminating the need for sugar or enzyme dosing while the fermentation is underway. Dosing during fermentation often requires associated controls such as real-time glucose monitoring, adjustments to dosing rate, and interim sterilizations of the glucose or enzyme feedstocks. The process disclosed here enables batch or simplified fed-batch operation of the fermenter inputs while getting fed-batch organism performance due to the continual release of sugars at optimal concentrations from enzymatic saccharification. Batch operation and/or fewer additions reduces the number opportunities for contamination due to incorrect or incomplete sterilization of process inputs.

### BRIEF DESCRIPTION OF THE DRAWINGS

Elements in the figures have not necessarily been drawn to scale in order to enhance their clarity and to improve the understanding of the various elements and embodiments of the invention. Furthermore, elements that are known to be common and well understood to those in the industry are not depicted in order to provide a clear view of the various embodiments of the invention. Thus, it should be understood that the drawings are generalized in form in the interest of clarity and conciseness.

FIG. 1 is a chart illustrating the reduced performance of microalgae at various glucose concentrations demonstrating inhibition at elevated glucose levels.

FIG. 2 is a chart illustrating the performance and stability of sugar levels provided by the methods of the present invention compared to the levels of traditional fed-batch glucose dosing.

FIG. 3A is a chart illustrating the growth and sugar profiles for a controlled saccharification run.

FIG. 3B is a chart illustrating the growth and sugar profile for a saccharification run using the corn/enzyme/algae batch SSF process of the present invention.

FIG. 3C shows a comparison of dry weights from uncontrolled and controlled saccharification.

FIG. 4 is a flow chart illustrating an exemplary method for producing biofuels according to a first preferred embodiment of the present invention.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may he practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

The present invention is directed to producing biofuels and other consumable products from biomass. These biofuels and consumable products may include food, feed, chemicals, fuels (i.e. renewable diesel, ethanol etc.) and the like, which for convenience are collectively referred to hereafter as biofuels. In particular, the invention is directed to processes intended to lower the cost of sugar feedstock and to simplify unit operations with improved contamination control when producing biofuels using a microalgae substrate. In addition, the invention may also be used to improve the quantity and/or quality of specific compounds produced by the algae, such as lipids (enhanced C18 profiles, for example), polysaccharides (enhanced rhamnose concentrations, for example), and proteins (increased quantity or improved nutritional profile, for example).

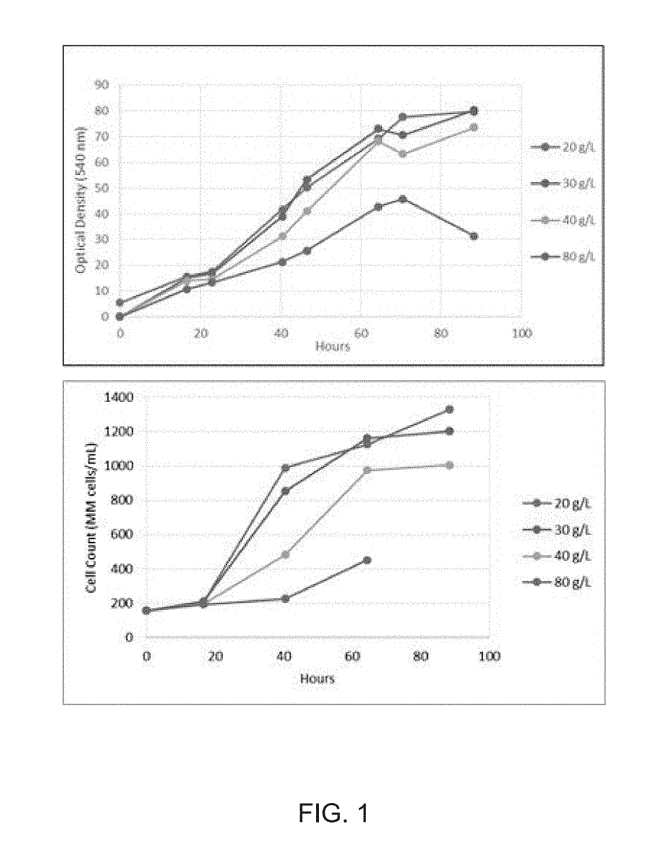

FIG. 1 illustrates the reduced performance of a microalgae substrate at various glucose concentrations. As shown, performance is similar at 20-30 g/L but decreases at higher glucose levels. Performance is reduced considerably at 80 g/L, with inhibition measured by both cell count and optical density.

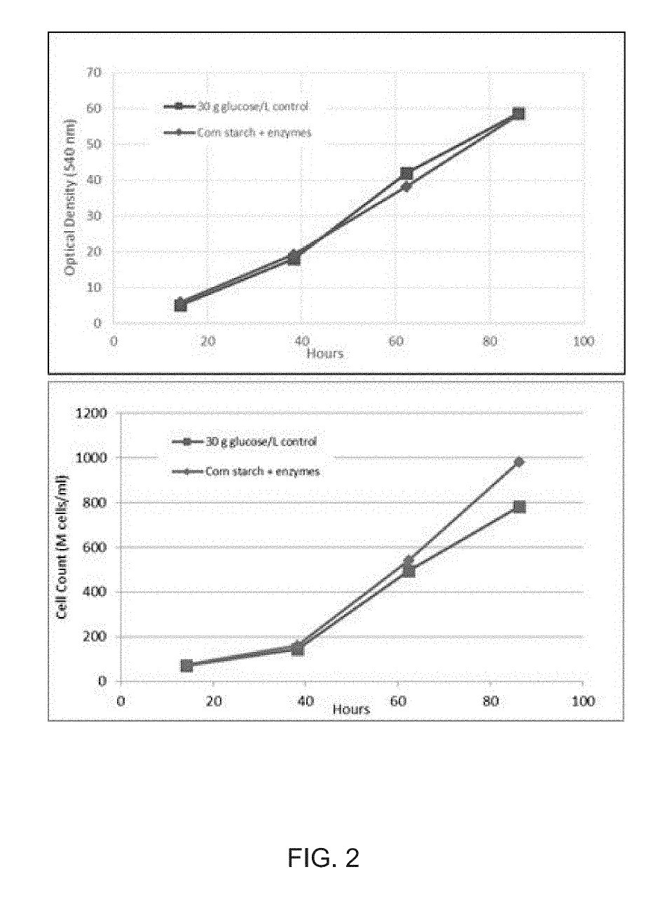

FIG. 2 shows the slightly enhanced sugar conversion rates achieved by a microalgae substrate when using the corn/enzyme/algae batch SSF process of the present invention versus a control fed-batch process with a target glucose level of 30 g/L.

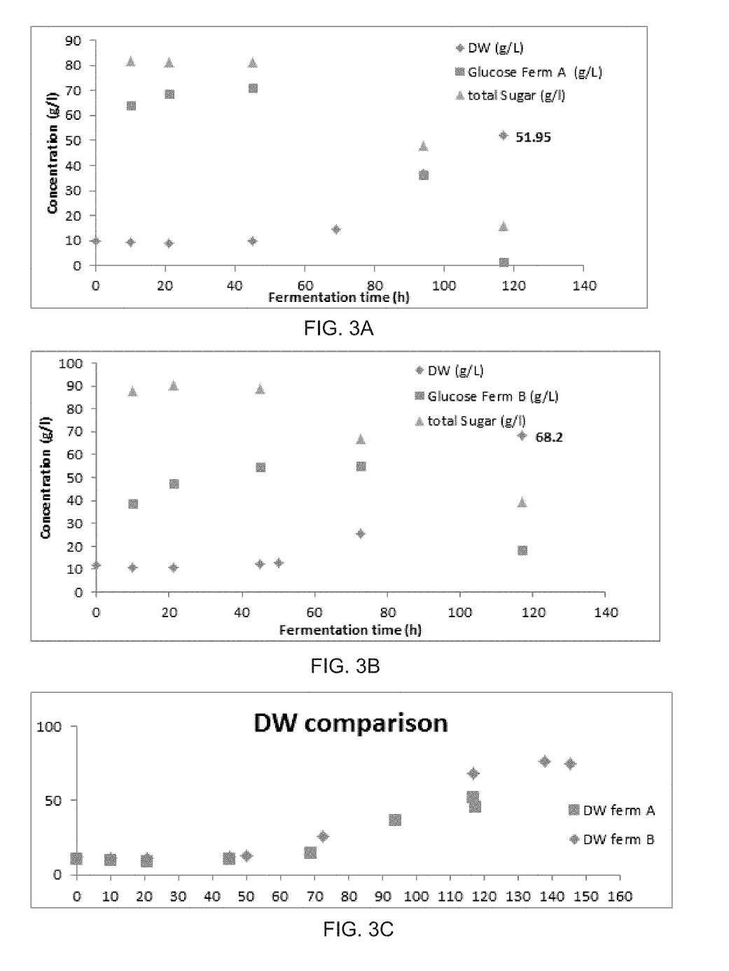

FIG. 3A shows the growth and sugar profiles for a controlled saccharification run using a fed-batch process. FIG. 3B shows the growth and sugar profile for a saccharification run using the corn/enzyme/algae batch SSF process of the present invention. As directly shown, the saccharification run using the corn/enzyme/algae batch SSF process of the present invention resulted in a 31.2% increase in the resulting dry weight of the processed and harvested material. Further, the resulting growth and sugar profiles for each run are almost identical in scale and shape.

FIG. 3C further shows a comparison of dry weights from the same uncontrolled and controlled saccharification trial as FIGS. 3A and 3B. As shown, the saccharification run using the corn/enzyme/algae batch SSF process of the present invention resulted in an increase in the resulting dry weight of the processed and harvested material.

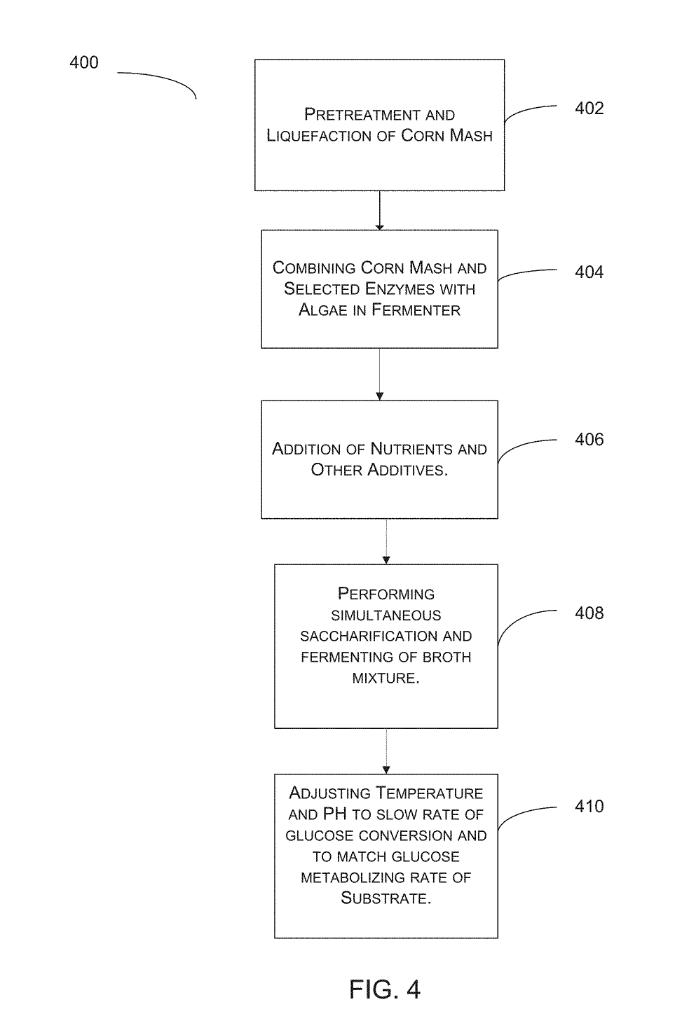

With referenced now to FIG. 4, an exemplary method 400 for producing biofuels according to a first preferred embodiment of the present invention will now be discussed. As shown, the preferred method of present invention preferably includes the pretreatment and liquefaction of a prehydrolysate feedstock 402. According to a preferred embodiment, the feedstock is preferably corn mash or similar corn derivative. Alternatively, the prehydrolysate feedstock may include any lignocellulosic biomass. According to a preferred embodiment, the pretreatment may include treatment using steam, with or without a catalyst. Alternatively, a steam explosion method may be used using an acid catalyst. In some embodiments, the pretreated carbohydrate feedstock is clarified using methods such as strainers (e.g. brush strainer), centrifugation (e.g. nozzle or decanter type centrifuges), or alternative liquid/solid separation methods to separate the liquefied carbohydrates from the solids. In such an embodiment, the liquid fraction would contain the carbohydrate feedstock to be used in subsequent saccharification and fermentation steps. In yet another embodiment, the prehydrolysate feedstock is also pretreated to enhance the release of nutrients for subsequent use by the algae, such as release of complex nitrogen compounds to supplement or replace external nitrogen addition requirements during fermentation.

As shown in step 404, once prepared, the pretreated corn mash is preferably combined with selected enzymes and a selected species of microalgae in a fermenting vessel. According to a preferred embodiment, the selected enzyme is preferably glucoamylase. Other enzymes may also be used depending on the biomass selected. Examples of alternative enzymes include: xylanase, amylase, lactase, diastase, sucrase; maltase; invertase; alpha-glactosidase and the like.

According to a further preferred embodiment, the preferred microalgae species is preferably from the Chlorella genus, including C. protothecoides, C. vulgaris, C. sorokiniana, C. saccharofila, and other Chlorella species. Alternatively, other microalgae species may be used such as Chlamydomonas reinhardtii, Chlorococcum littorale, Platymonas subcordiformis, Anabaena, Nostoc muscorum, N. spongiaeforme, Westiellopsis prolifica, Oscillotoria Miami BG7 or Aphanothece halophytico.

With reference to Step 406, after or with the combining of the biomass, enzymes and microalgae in Step 404, additional nutrients may be further added as needed. For instance, nitrogen and phosphorous may be added to assist in microalgae growth. According to a preferred embodiment, nutrient-replete conditions can be maintained in several alternative ways, including: adding all of the needed nutrients at the beginning of a fermentation; by interim bolus additions during the course of the fermentation; or through continuous additions to maintain target levels in the fermentation broth.

With reference to Step 408, the broth mixture is then fermented to produce microalgal biomass. Preferably, the enzymatic hydrolysis and fermentation are performed simultaneously, i.e., simultaneous saccharification and fermentation (SSF).

With reference to Step 410, during the SSF process, the temperature and pH of the broth mixture are preferably measured and adjusted to slow the rate of glucose conversion and to match the glucose metabolizing rate of the microalgae. According to a preferred embodiment, for the preferred enzyme/algae combination of glucoamylase and Chlorella protothecoides, the preferred target pH is preferably in the range of 5.0-6.5 with a temperature in the range of 24-32° C.

According to a further aspect of the present invention, sugar release kinetics can be controlled in several ways, including maintaining sub-optimum pH and temperature for a given enzyme, controlling enzyme dosing rates, or through the use of slower-acting enzymes that are designed for the pH and temperatures that are optimal for the organism in production.

In alternative embodiments, other process streams from the co-located facility may be used to enhance the quality or quantity of algae produced with or without the SSF process. For example, the carbohydrates in corn syrup or scrubber condensate may be utilized by the algae in a fermenter or alternative bioreactor (including photobioreactors) to increase biomass quantity or quality.

The algal biomass or algal product may he processed into the final form using methods appropriate for the specific market. In one embodiment, algal biomass may be dried and/or mixed with corn solids to improve the pigment or protein content and quality for aquafeed, animal feed, or food applications.

### EXAMPLE

An example of the present invention was carried out in 30 L fermenters. All inputs to the fermenter were sterilized prior to inoculation with the target organism. Liquefied corn mash was sourced from a corn-starch-to-ethanol plant, clarified by filtration and centrifugation, sterilized in the fermenter for 30 min. All nutrients and antifoam were sterilized prior to addition at 121° C. for 30-60 min and aseptically transferred to the fermenter. Commercially-available glucoamylase was filter-sterilized prior to addition, and then live algal culture was transferred to the fermenter to commence fermentation.

Temperature in the reactor was controlled at a setpoint between 22-32° C. and a setpoint between 5.3-6.3. In contrast, the temperature optimum of glucoamylase is often ˜60° C. and a pH optimum of 4.0. The fermenter was jacketed with automated temperature control and equipped with aeration and agitation to maintain aerobic conditions throughout the fermentation.

Results of the trial showed that densities (in g/L) were 31% higher with controlled. enzyme activity after 117 h of fermentation time compared to a fermenter under similar conditions but elevated glucose levels due to higher glucoamylase activity early in the fermentation. Both fermentations exceeded 50 g/L in biomass concentration. The starting concentration of glucoamylase was 4× higher in the underperforming fermenter, resulting in glucose concentrations that were 30% higher after 45 h of elapsed fermentation time in the underperforming fermenter.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not he limited by this detailed description, but by the claims and the equivalents to the claims appended hereto.

What is claimed is:

1. A process for producing biofuels, wherein the process comprises:
pretreatment and liquefaction of a prehydrolysate feedstock;
initiating enzymatic hydrolysis by combining the prehydrolysate feedstock with one or more enzymes and one or more species of microalgae in a fermenting vessel to produce a reaction mixture;
adding nutrients to the reaction mixture to assist microalgae growth;
fermenting the reaction mixture to produce microalgal biomass, wherein the enzymatic hydrolysis and fermentation are performed simultaneously; and
measuring and adjusting the temperature and pH of the reaction mixture during the simultaneous enzymatic hydrolysis and fermentation; wherein the temperature and pH of the reaction mixture are measured and adjusted to slow the rate of glucose conversion and to match the glucose metabolizing rate of the microalgae.

2. The process of claim 1, wherein the feedstock is a corn derivative.

3. The process of claim 2, wherein the pretreatment comprises applying steam.

4. The process of claim 3, wherein the pretreatment comprises a steam explosion treatment using an acid catalyst.

5. The process of claim 2, wherein at least one selected enzyme is glucoamylase.

6. The process of claim 5, wherein at least one selected enzyme is selected from the group of enzymes which includes: xylanase, amylase, lactase, diastase, sucrose, maltase, invertase, and alpha-glactosidase.

7. The process of claim 5, wherein at least one selected microalgae is from the Chlorella genus.

8. The process of claim 7, wherein at least one selected microalgae is selected from the group of microalgae which includes: Chlamydomonas reinhardtii, Chlorococcum littorale, Platymonas subcordiformis, Anabaena, Nostoc muscorum, N. spongiaeforme, Westiellopsis prolifica, Oscillotoria Miami BG7 and Aphanothece halophytico.

9. The process of claim 7, wherein the step of adding nutrients to the reaction mixture to assist microalgae growth comprises adding nitrogen and phosphorous.

10. The process of claim 9, wherein the selected enzyme is glucoamylase and the selected microalgae species is Chlorella protothecoides; further wherein the pH is adjusted to a range of 5.0-6.5 and the temperature is adjusted to a range of 24-32° C.
